(19) United States
(12) Patent Application Publication
(10) Pub. No.: US 2006/0013408 A1
(43) Pub. Date: Jan. 19, 2006

(54) AUDIO DEVICE WITH ACTIVE NOISE CANCELLATION

(76) Inventor: Yi-Bing Lee, Taipei (TW)

Correspondence Address: JIANQ CHYUN INTELLECTUAL PROPERTY OFFICE, 7 FLOOR-1, NO. 100, ROOSEVELT ROAD, SECTION 2, TAIPEI, 100, TW

(21) Appl. No.: 10/905,967

(22) Filed: Jan. 28, 2005

(30) Foreign Application Priority Data
Jul. 14, 2004 (TW) 93120944

(51) Int. Cl. A61F 11/06; G10K 11/16; H03B 29/00

(52) U.S. Cl. 381/71.6; 381/72

(57) ABSTRACT

An audio device with active noise cancellation (ANC) for coupling to a stereo headset is provided. The audio device comprises an audio processor and an analog ANC control module. The audio processor processes audio data for playback. It optionally can also comprise digital ANC function for tracking and canceling narrow band noise. The analog ANC control module actively cancels wide band noise in the stereo headset based on an audio feedback signal. The audio device further comprises a power amplifier and a feedback processor. The power amplifier is coupled to the output of the audio processor and the analog ANC control module. The power amplifier amplifies the output from the audio processor and the analog ANC control module and sends the amplified signal to the stereo headset. The feedback processor receives the noise from the stereo headset and generates an signal to the analog ANC control module.

### CROSS-REFERENCE TO RELATED APPLICATION

This application claims the priority benefit of Taiwan application serial no. 93120944, filed Jul. 14, 2004.

### BACKGROUND OF THE INVENTION

1. Field of the Invention

The present invention relates to an audio device. More particularly, the present invention relates to an audio device equipped with an active noise cancellation function.

2. Description of the Related Art

Portable audio device has been in our everyday life for a long time. The popularity had experienced a boom in mid 70's when Sony began marketing their famous “Walkman” line of product, it's so popular that the product name “Walkman” even became a synonym of portable audio device. During that period of time, such device relied on magnetic cassette tape as major storage media.

In the early 1990s, the audio industry has been affected by the digital revolution so that various type of digital audio formats start to infiltrate the mechanical/analog media market. As the prices of digital processing chips fall and the fabrication process and hardware architecture continue to improve, more and more portable audio equipment uses digital hardware. With time, digital portable audio devices such as MP3, WMA players have become the mainstream products in the market. However, it does not matter if the portable audio device operates in an analog or digital format, the environment may sometimes render the device unusable. For example, in a noisy public place, the user can hardly enjoy a musical playback by listening through a headset. In brief, noise in the environment is one big problem for the listener of a portable audio device.

FIG. 1 is a block diagram showing a conventional digital audio device having an active noise cancellation headset. To combat noise problem, an active noise cancellation headset (ANC) headset has been developed. As shown in FIG. 1, the ANC headset 120 has an active noise cancellation (ANC) control circuit 110. The ANC headset 120 is also coupled to an audio device 130. The audio device 130 comprises an audio processor 132 for processing audio data and providing audio playback signals to the speaker 122 inside the headset 120 and a power supply unit 134 for providing the power necessary for driving the audio processor 132.

The ANC headset 120 further comprises a receiver 124 for sensing any noise inside the ANC headset 120 and submitting the noise signal to the ANC control circuit 110. The ANC control circuit comprises analog ANC control module 112, a power amplifier 116 and a feedback processor 118. The analog ANC control module 112 actively cancels the noise inside the ANC headset 120 according to the output from the ANC headset 120. The ANC control circuit 110 also includes a power supply unit 114 for providing the power necessary for driving the analog ANC control module 112.

Although the conventional ANC control headset 120 is able to reduce noise interference in most audio playback equipment such as MP3, the ANC headset 120 is rather bulky. Furthermore, the provision of a power supply unit 114 inside the ANC headset 120 for driving the control circuit also adds some weight to the ANC headset 120. One other major defect of current ANC headset based on analog circuit is that, the frequency response of the headset can be heavily affected by the onset of ANC circuit. The playback quality differs drastically upon switching on/off of ANC function. This interaction between ANC circuit and headset frequency response has been one of the major problem in introducing ANC function into audio devices.

### SUMMARY OF THE INVENTION

Accordingly, at least one objective of the present invention is to provide an audio device with active noise cancellation function integrated so that when coupling with a simple headset/microphone combination, the effect of noise on the music listening can be minimized.

At least a second objective of the present invention is to provide an audio device integrated with active noise cancellation that can reduce the volume and weight of an ANC headset by sharing power supply and certain circuit.

At least a third objective of the present invention is to provide an digital audio device integrated with active noise cancellation function, so that the interaction of the audio playback and ANC function can be tuned together so that the variation of headset frequency response can be minimized upon switching ANC function.

To achieve these and other advantages and in accordance with the purpose of the invention, as embodied and broadly described herein, the invention provides an audio device with active noise cancellation for driving a stereo headset. The audio device comprises an audio processor and an analog active noise cancellation (ANC) control module. The audio processor processes audio data and drives the stereo headset. The analog ANC control module actively cancels any noise generated inside the stereo headset according to a feedback signal. The audio device further comprises a power amplifier and a feedback processor. The power amplifier is coupled to the output of the audio processor and the analog ANC control module. The power amplifier amplifies the output from the audio processor and the analog ANC control module and submits the amplified signals to the stereo headset. The feedback processor receives noise signals detected from the stereo headset and provides a feedback audio signal to the analog ANC control module.

In the embodiment of the present invention, the audio device further comprises a power supply unit for providing electrical power to the audio processor and the ANC control module.

Preferably, the stereo headset further comprises an audio source and a detecting source. The audio source is coupled to the power amplifier for audio playback. The detecting source is used for sensing the noise inside the stereo headset and outputting the detected noise signal to the feedback processor. In the embodiment of the present invention, the audio source is a speaker and the detecting source is an microphone.

The present invention also provides an audio device with active noise cancellation that can be coupled to a stereo headset for driving the headset. The audio device comprises a digital audio processor for processing audio data and driving the stereo headset to produce source. Furthermore, the digital audio processor also has a active noise cancellation (ANC) function. Hence, the audio processor can actively cancel the inference from noise inside the stereo headset. In addition, the audio device further comprises a power amplifier and a feedback processor. The power amplifier is coupled to the output of the audio processor for amplifying the output from the audio processor and submitting the amplified signal to the stereo headset. The feedback processor receives the noise signal detected by stereo headset and generates a feedback audio signal to the audio processor.

In the embodiment of the present invention, the digital ANC function is implemented in digital processing format.

In an alternative embodiment, the present invention further comprises an analog ANC control module having an output coupled to the power amplifier. The analog ANC control module receives the feedback audio signal and actively cancels the interference from the noise having a second frequency bandwidth inside the stereo headset.

In addition, the power supply unit also provides electrical power to the ANC control module.

In brief, the ANC control module of the present invention minimizes the effect of noise on the user of a stereo headset. The present invention also permits the incorporation of a digital ANC unit inside the audio processor so that the noise within a narrow bandwidth can also be eliminated. Furthermore, the analog ANC control module is not set up within the stereo headset so that the volume of the stereo headset can be reduced. In addition, both the analog ANC control module and the audio processor use the same power supply unit. Hence, overall weight of the audio device can be reduced.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a block diagram showing a conventional digital audio device having an active noise cancellation headset.

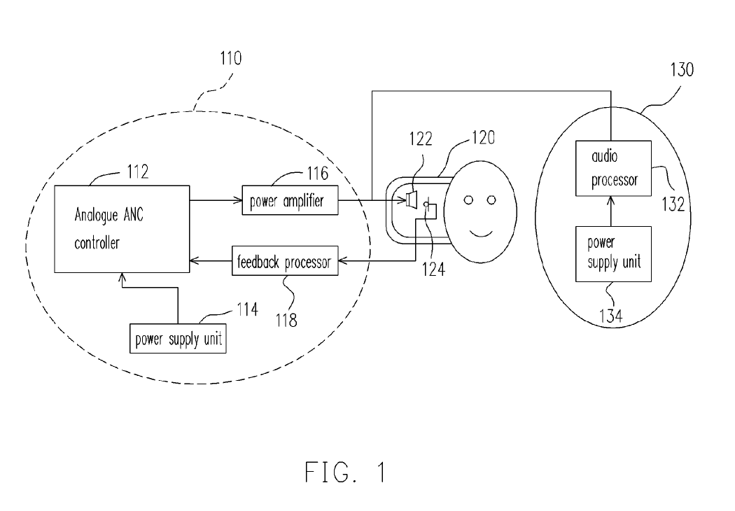

FIG. 2 is a block diagram showing an audio device having active noise cancellation function according to a first embodiment of the present invention.

FIG. 3 is a block diagram showing an audio device having active noise cancellation function according to a second embodiment of the present invention.

FIG. 4 is a block diagram showing an audio device having active noise cancellation function according to a third embodiment of the present invention.

### DESCRIPTION OF THE PREFERRED EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

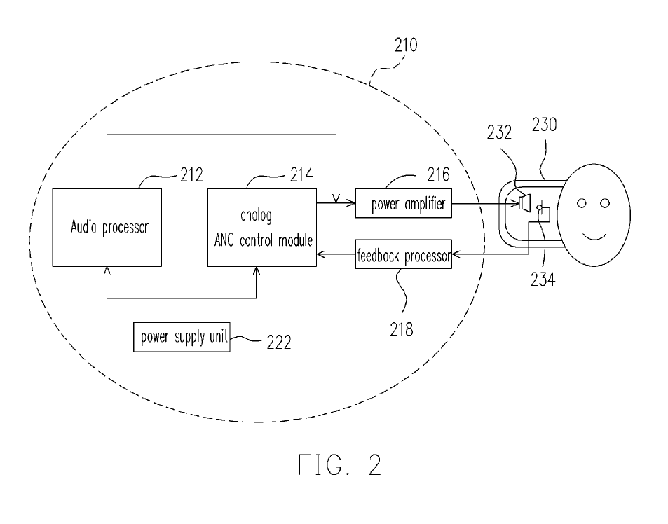

FIG. 2 is a block diagram showing an audio device having active noise cancellation function according to a first embodiment of the present invention. As shown in FIG. 2, the present invention provides an audio device 210 suitable for driving a stereo headset 230. The present invention may apply to a thumb disk with music playing function. Moreover, the stereo headset 230 is a unit having a unidirectional playback earplug and a feedback receiver but no conventional ANC circuit inside. Inside the audio device 210, the output from an audio processor 212 and an analog audio noise cancellation (ANC) control module 214 are transmitted to the stereo headset 230 through a power amplifier 216. Meanwhile, the stereo headset 230 also transmits signals corresponding to the noise detected inside the headset 230 to a feedback processor 218 inside the audio device 210.

As shown in FIG. 2, the audio processor 212 processes audio data such as MP3 music files. The output from the audio processor 212 and the analog ANC control module 214 are both sent to the power amplifier 216. The power amplifier 216 amplifies the output from the audio processor 212 and the analog ANC control module 214 before sending the amplified output to the stereo headset 230 for playing the audio data processed by audio processor 230 on the stereo headset 230.

In general, the stereo headset 230 comprises an audio source 232 and a detecting source 234. The audio source 232 receives the output from the power amplifier 216. The detecting source 234 senses the noise inside the stereo headset 230 and transmits a noise-related signal to the feedback processor 218. In the present embodiment, the audio source 232 can be a speaker and the detecting source 234 can be an microphone, for example.

After the power amplifier 216 has amplified the output from the audio processor 212, the amplified signal is transmitted to the stereo headset 230 for a playback to listener. In the meantime, the detecting source 234 senses the noise inside the stereo headset 230 and transmits a corresponding signal to the feedback processor 218. The noise within the stereo headset 230 can be any sound produced by the surrounding. After picking up an output signal from the detecting source 234, the feedback processor 218 amplifies the output signal to generate and provide a feedback audio signal to the analog ANC control module 214.

On receiving the feedback audio signal from the feedback processor 218, the analog ANC control module 214 will produce a noise cancellation signal to the power amplifier 216 through a few simple internal transfer circuits. Thus, the power amplifier 216 can amplify both the noise cancellation signal and the output of the audio processor 212 before sending them to the stereo headset 230. Ideally, the noise cancellation signal is out of phase with the audio noise signal. In other words, the noise cancellation signal is able to suppress the noise inside the stereo headset 230. In practice, due to imperfection in the circuit devices, the noise cancellation signal can hardly be exactly out of phase with the audio noise signal. Yet, no matter what, the stereo headset 230 will at least output a portion noise cancellation signal to minimize the noise interference and improve the quality of playback sound.

Furthermore, the present invention may comprise a power supply unit 222 such as a battery module coupled to the audio processor 212 and the analog ANC control module 214 for providing necessary power for driving the two units.

In the present embodiment, the audio device 210 has incorporated the ANC function so that the stereo headset on the user's head can be simplified to include a typical playback portion and a simple feedback detecting source 234 such as an microphone only. In other words, the design is able not only to reduce the weight of the headset but also to cancel environmental noise.

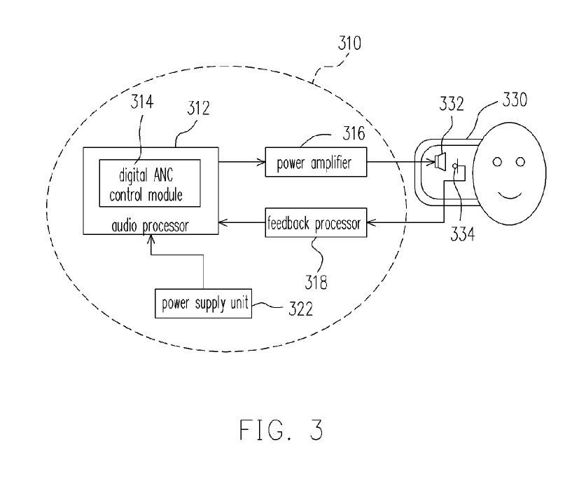

FIG. 3 is a block diagram showing an audio device having active noise cancellation function according to a second embodiment of the present invention. As shown in FIG. 3, an audio device 310 is provided to eliminate interfering noise from a narrow frequency band. The audio processor 312 inside the audio device 310 has a digital ANC control module 314. The digital ANC control module 314 provides the audio processor 312 with a digital ANC function for eliminating noise interference from a narrow frequency band.

Similarly, the audio device 310 of the present embodiment matches with the operation of a stereo headset 330. Inside the audio device 310, the output of the audio processor 312 is coupled to the power amplifier 316. After amplifying the output from the audio processor 312, the power amplifier 316 transmits the amplified output signal to the stereo headset 330. In addition, the audio device 310 further comprises a feedback processor 318 for receiving noise signal from the stereo headset 330 and transmitting a feedback audio signal to the audio processor 312.

Because the audio processor 312 encloses the digital ANC control module 314, the digital ANC control module 314 will provide the output from the audio processor 312 with a noise cancellation signal as shown in the first embodiment. Therefore, when the stereo headset 330 is playing back audio message, the noise cancellation signal will cancel some of the interference noise resulting from a narrow frequency band. In the present embodiment, the digital ANC control module 314 can effect a noise cancellation by performing a digital signal processing operation.

Furthermore, the audio device 310 may comprises a power supply unit 322 coupled to the audio processor 312 for providing electrical power to drive the audio processor 312.

In the present embodiment, the stereo headset 330 comprises an audio source 332 and a detecting source 334. The audio source 332 receives an output of the power amplifier 316. The output of the detecting source 334 is similarly coupled to the feedback processor 318. Since the audio source 332 and the detecting source 334 inside the stereo headset 330 operate in a manner similar to the stereo headset 230 in the first embodiment, detailed description is omitted.

The present embodiment is very similar to the first embodiment in that the audio device 310 has incorporated the ANC function. Hence, the stereo headset on the user's head can be simplified to include a typical playback portion and a simple feedback detection unit 334 such as a microphone only. All in all, the design is able not only to reduce the weight of the headset but also to cancel environmental noise.

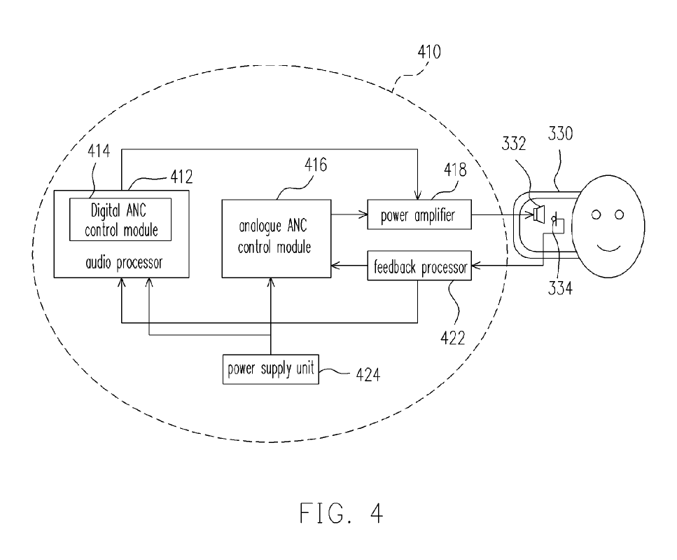

FIG. 4 is a block diagram showing an audio device having active noise cancellation function according to a third embodiment of the present invention. As shown in FIG. 4, the audio device 410 in the present embodiment is a hybrid between the audio device 210 of the first embodiment and the audio device 310 of the second embodiment of the present invention. This arrangement is particularly suitable for removing wide as well as narrow band interfering noise content simultaneously. The audio device comprises an audio processor 412 and an analog ANC control module 416. The output from the audio processor 412 and the analog ANC control module 416 are coupled to the power amplifier 418. The output of feedback processor 416 is coupled to the audio processor 412 and the analog ANC control module 416. The audio processor 412 also encloses a digital ANC control module 414 so that the audio processor 412 has the capacity to perform typical digital ANC function. The audio device 410 further comprises a power supply unit 424 for providing electrical power to drive the audio processor 412 and the analog ANC control module 416.

The third embodiment is actually a combination of the operating principle and performance level of the first and the second embodiment. Aside from canceling the multi-band noise described in the first embodiment, the audio device 410 of the present embodiment also eliminates the narrow band noise described in the second embodiment as well. Hence, the third embodiment of the present invention is able to provide a better audio noise cancellation than the previous two embodiments.

In summary, major advantages of the present invention at least includes:

1. The ANC circuit is installed inside the audio device instead of the stereo headset. Hence, overall volume of the ANC stereo headset is reduced.

2. The analog ANC control module uses simple logic circuit connection so that simple circuits can be deployed to increase yield and system reliability and lower production cost.

3. The phase difference of the audio signal due to a time delay in the noise feedback mechanism of the ANC stereo headset can be used to cancel in-head effect as well.

4. An ANC control module can be disposed inside the audio processor for an effective cancellation of noise interference from a narrow frequency band.

5. Both the audio processor and the analog ANC control module are driven by a single power supply unit so that weight can be reduced.

6. The audio device may integrate an audio processor having a digital ANC control module therein and an analog control module together so that interference from both broad band and narrow band noise can be attenuated.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. An audio device with active noise cancellation (ANC) for driving a stereo headset, the audio device comprises:
a digital audio processor for processing audio data and driving the stereo headset to emit sound;
an analog ANC control module for actively canceling the noise inside the stereo headset according to a feedback audio signal;
a power amplifier coupled to the output of the audio processor and the analog ANC control module for amplifying the output from the audio processor and the analog ANC control module and transmitting the amplified output to the stereo headset; and
a feedback processor for receiving the noise sensed by the stereo headset and providing a feedback audio signal to the analog ANC control module.

2. The audio device of claim 1, wherein the audio device further comprises a unified power supply unit for providing electrical power to drive the audio processor and the ANC control module.

3. The audio device of claim 1, wherein the stereo headset further comprises:
an audio source coupled to the power amplifier for playing back sound messages; and
a detecting source for sensing the noise around the stereo headset and sending a corresponding signal to the feedback processor.

4. The audio device of claim 3, wherein the audio source comprises a speaker.

5. The audio device of claim 3, wherein the detecting source comprises an microphone.

6. The audio device of claim 1, wherein the audio device comprises an audio content storage with audio playback function.

7. An audio device with active noise cancellation (ANC) for operating a stereo headset, the audio device comprising:
an audio processor for processing audio data and driving the stereo headset to emit sound, wherein the audio processor also has a digital ANC function for actively canceling the interference of a first frequency band noise originated inside the stereo headset according to a feedback audio signal;
a power amplifier for receiving the output signal from the audio processor and sending the amplified output signal to the stereo headset; and
a feedback processor for receiving the noise signal sensed by the stereo headset and providing a feedback audio signal to the audio processor.

8. The audio device of claim 7, wherein the audio device further comprises a power supply unit for providing electrical power to the audio processor.

9. The audio device of claim 7, wherein the digital ANC function within the stereo headset is implemented through digital signal processing operations. The digital ANC is aimed at tracking and canceling narrow band noise.

10. The audio device of claim 7, wherein the audio device further comprises an analog ANC control module having an output terminal coupled to the power amplifier such that the analog ANC control module receives the feedback audio signal and actively cancel the interference of wide band noise originated inside the stereo headset according to the feedback audio signal.

11. The audio device of claim 10, wherein the audio device further comprises a power supply unit for providing electrical power to drive the audio processor and the analog ANC control module.

12. The audio device of claim 7, wherein the stereo headset comprises:
an audio source coupled to the power amplifier for playing back sound messages; and
a detecting source for sensing the noise around the stereo headset and sending a corresponding signal to the feedback processor.

13. The audio device of claim 12, wherein the audio source comprises a speaker.

14. The audio device of claim 12, wherein the detecting source comprises an microphone.

15. The audio device of claim 7, wherein the audio device comprises an audio content storage with audio playback function.
